(19) United States
(12) Patent Application Publication
(10) Pub. No.: US 2019/0175391 A1
(43) Pub. Date: Jun. 13, 2019

(54) FACIAL TEMPERATURE PACK HOLDER

(71) Applicant: Sharon R. Shemtov, Peachtree Corners, GA (US)

(72) Inventor: Sharon R. Shemtov, Peachtree Corners, GA (US)

(21) Appl. No.: 15/839,696

(22) Filed: Dec. 12, 2017

(51) Int. Cl. A61F 7/02; A61F 7/10

(52) U.S. Cl. CPC A61F 7/02; A41D 13/1161; A61F 7/10

(57) ABSTRACT

A facial temperature pack holder may comprise a housing shaped similarly to earmuffs, wherein the housing has a top portion, a first side portion, and a second side portion, and wherein the housing provides for one or more of a top pocket, a first side pocket, and a second side pocket, and wherein one or more of the top pocket, first side pocket, and/or second side pocket are configured to contain at least one temperature pack. The present invention may also provide for at least one flexible connection fixedly connected to the top portion and one or more of the first side portion and/or the second side portion. The present invention may further comprise an interior surface or lining that may be configured to impart pleasing tactile properties. The present invention may also provide for one or more elements of webbing that may line one or more outside edges of the housing.

### CROSS-REFERENCE TO RELATED APPLICATION

Not applicable.

### TECHNICAL FIELD

The present invention relates generally to the field of therapeutic devices and more specifically relates to a warm, hot, cold, or cool pack and holder configured for use on a face.

### BACKGROUND

Persons who have had facial or oral surgery tend to have facial swelling, bruising, or other tissue injury. As such, it is beneficial for these persons to apply cold packs to these facial areas as treatments. However, these persons are often required to hold the cold pack against the person's face. This design limits the effectiveness of such treatments, because the person's arm can become tired. Other persons may not have the ability to lift their arm to that position or hold their arm to that position for a long enough time. Therefore, a need exists for a solution that can hold the cold pack against the person's face.

Various attempts to meet this need have been made. One such attempt can be seen with respect to U.S. Patent Application Publication No. 2004/0138729, incorporated by reference in its entirety herein, which generally discloses a mask that has strip-shaped areas for applying heat or cold to a person's face. While this disclosure does provide for a facial cold pack solution, its mask design is inconvenient and its cold pack application sites are limited.

Another attempt can be seen with respect to U.S. Pat. No. 6,582,383, incorporated by reference in its entirety herein, which generally discloses a strip-shaped hot or cold pack application device. While this disclosure does provide for a more precise application of cold to a user's face, its strip design limits its utility.

Yet another attempt can be seen with respect to U.S. Pat. No. 6,451,046, incorporated by reference in its entirety herein, which generally discloses a head bandage-type design temperature pack application device. While this disclosure does provide for the flexibility of attaching the strap more towards the user's nose and front of the face, its under-the-chin design limits its utility.

Various attempts have been made to solve the problems which may be found in the related art but have thus far been unsuccessful. A need exists for a new facial temperature pack holder to avoid the above-mentioned problems.

### SUMMARY OF THE INVENTION

It is to be understood that in the present disclosure, all embodiments are provided as illustrative and non-limiting representatives of many possible embodiments. In addition, the terms “is,” “can,” “will,” and the like are herein used as synonyms for and interchangeable with terms such as “may,” “may provide for,” and “it is contemplated that the present invention may” and so forth.

Furthermore, all elements listed by name, such as an ice pack, cold compress, hook and loop fastener, earmuffs, etc., are herein meant to include or encompass all equivalents for such elements. For example, in addition to a “cold compress,” any pack or compress capable of retaining and/or delivering either hot or cold is also contemplated by the present invention. Such equivalents are contemplated for each element named in its particular herein.

The present invention relates to a facial temperature pack holder that overcomes the deficiencies of the prior art. It is contemplated that the present invention may provide an earmuff-shaped temperature pack apparatus configured for use on a person's face.

For purposes of summarizing, certain aspects, advantages, and novel features of the present invention are provided herein. It is to be understood that not all such aspects, advantages, or novel features may be provided in any one embodiment. Thus, the disclosed subject matter may be embodied or carried out in a manner that achieves or optimizes one aspect, advantage, or novel feature or group of features without achieving all aspects, advantages, or novel features as may be taught or suggested.

In view of the foregoing disadvantages inherent in the known art, the present invention provides a novel facial temperature pack holder. The general purpose of the present invention, which will be described subsequently in greater detail, is to provide a facial temperature pack holder apparatus that enables a user to apply cold (or heat) to the user's face so as to alleviate pain, reduce swelling, or achieve other desired outcomes. The features of the invention which are believed to be novel are particularly pointed out and distinctly claimed in the concluding portion of the specification. These and other features, aspects, and advantages of the present invention will become better understood with reference to the following drawings and detailed description.

In one embodiment, the present invention may provide for a facial device that may be used on recent patients who have come out of a facial or oral surgery, and that may provide reinforcement when icing, during their recovery process. Similar in appearance to ear muffs, but instead of an ear attachment, the present invention may provide for attachments that may extend from a headpiece and that may cover the cheeks and/or the sides of users faces. Inside one or more pockets of the present invention, a user may insert one or more cold compress packs, cool compress packs, warm compress packs, hot compress packs, ice packs, temperature-changing packs, or other type of temperature-delivering packs (collectively, “temperature packs”). The present invention's structure may be configured such that that users of the device can better ice or warm their cheeks and faces using the application of a temperature pack, thus promoting a speedy recovery time. In some embodiments, the present invention may be adjustable, and may fit an array of head proportions and facial shapes.

More specifically, in some embodiments, the present invention may provide for a facial temperature pack holder, comprising a housing having at least a top portion, a first side portion, and a second side portion. In some embodiments, at least one top pocket may be fixedly connected to the top portion, at least one first side pocket may be fixedly connected to the first side portion, and at least one a second side pocket may be fixedly connected to the second side portion. Moreover, at least one temperature pack may be positioned within the first side pocket.

In some embodiments, the at least one temperature pack may be removably positioned within the first side pocket. In some embodiments, the at least one temperature pack may be removably positioned within one or more of the top pocket, the first side pocket, and/or the second side pocket.

The present invention may further comprise at least one flexible connection fixedly connected to the top portion and the first side portion and/or the second side portion.

In some embodiments, the first side portion and/or the second side portion may be configured to cover at least part of a facial cheek.

In some embodiments, the housing may be shaped like earmuffs wherein the shape of the housing is configured for use with facial cheeks. In other embodiments, the housing may be shaped like headphones, wherein the shape of the housing is configured for use with ears.

In some embodiments, the at least one temperature pack may be permanently affixed within one or more of the top pocket, the first side pocket, and/or the second side pocket.

In some embodiments, the present invention may further comprise an interior lining on the inside of the housing. In some embodiments, the present invention may further comprise at least one webbing element disposed along at least one outside edge of the housing.

The unique features of the present invention may provide one or more valuable benefits for one or more consumers. For example, it may allow users to feel less pain following their facial or oral procedures by compressing their cheeks and allowing ice to better sit on their faces, without the need of having to wrap it in a towel, or use additional compresses. Additionally, the present invention may fit a variety of head proportions and facial shapes, for users of all sizes. Furthermore, its convenient design may allow users to be hands-free while their face is recovering, and users can wear the device while lying in bed, resting on the sofa, or even sitting upright. Moreover, due to its unique design, the present invention may facilitate the use of headphones with electronic devices like tablets, personal computers, or mobile phones while the user is enjoying the other benefits of the present invention.

### BRIEF DESCRIPTION OF THE DRAWINGS

These and other features, aspects and advantages of the present invention will become better understood with regard to the following description, appended claims, and accompanying figures where:

FIG. 1 shows a front perspective view of a facial temperature pack holder in accordance with one embodiment of the invention.

FIG. 2 shows a front view of a facial temperature pack holder in accordance with one embodiment of the invention.

FIG. 3 shows a side view of a facial temperature pack holder in accordance with one embodiment of the invention.

FIG. 4 shows a top perspective view of a facial temperature pack holder in accordance with one embodiment of the invention.

FIG. 5 shows a top perspective view of a facial temperature pack holder in accordance with one embodiment of the invention.

### DETAILED DESCRIPTION

The present invention overcomes the limitations of the prior art by providing a new and more effective facial temperature pack holder.

All dimensions specified in this disclosure are by way of example only and are not intended to be limiting. Further, the proportions shown in these Figures are not necessarily to scale. As will be understood by those with skill in the art with reference to this disclosure, the actual dimensions and proportions of any embodiment or element of an embodiment disclosed in this disclosure will be determined by its intended use.

It is to be understood that the drawings and the associated descriptions are provided to illustrate potential embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

As used in this disclosure, except where the context requires otherwise, the term “comprise” and variations of the term, such as “comprising”, “comprises” and “comprised” are not intended to exclude other additives, components, integers or steps.

In the following description, specific details are given to provide a thorough understanding of the embodiments. However, it will be understood by one of ordinary skill in the art that the embodiments may be practiced without these specific detail. Well known features, elements or techniques may not be shown in detail in order not to obscure the embodiments.

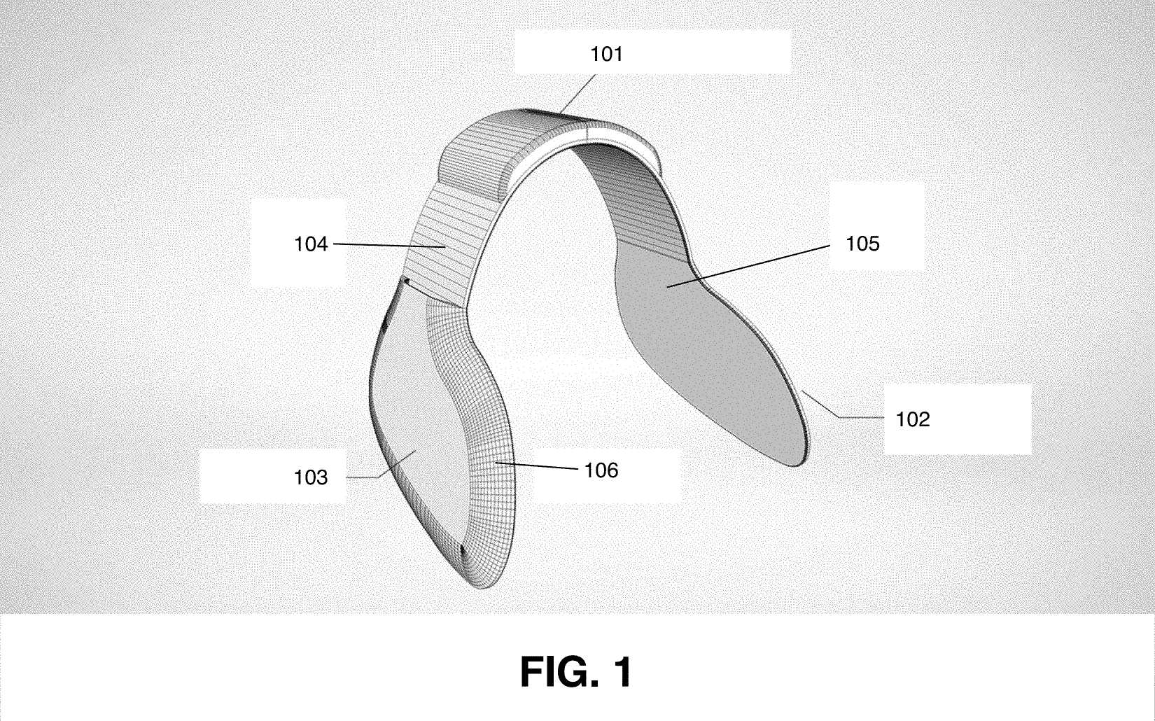

Turning attention to FIG. 1, a front perspective view of a facial temperature pack holder in accordance with one embodiment of the invention is shown. In the embodiment depicted, a viewer may perceive top pocket 101, first side pocket 102, second side pocket 103, flexible connection 104, interior surface 105, and webbing 106.

In some embodiments, one or more of top pocket 101, first side pocket 102, and/or second side pocket 103 may be configured to removably retain one or more temperature packs (not shown). Although the present invention in FIGS. 1-5 has been shown with the same portions labeled either first side pocket 102 or second side pocket 103, in embodiments wherein only one side pocket is provided (such embodiments, not shown), that side pocket is first side pocket 102.

It is contemplated that one or more of top pocket 101, first side pocket 102, and/or second side pocket 103 may have an open design, or one or more of top pocket 101, first side pocket 102, and/or second side pocket 103 may have a closeable design. In the open design, a user may insert and remove a temperature pack in one or more of top pocket 101, first side pocket 102, and/or second side pocket 103 without having to open one or more of top pocket 101, first side pocket 102, and/or second side pocket 103. Correspondingly, in an open design, a temperature pack may fall out of or otherwise be held less securely within one or more of top pocket 101, first side pocket 102, and/or second side pocket 103.

In some embodiments, one or more of top pocket 101, first side pocket 102, and/or second side pocket 103 may have a closeable design, such that one or more of top pocket 101, first side pocket 102, and/or second side pocket 103 may be opened so that a temperature pack may be inserted, and then one or more of top pocket 101, first side pocket 102, and/or second side pocket 103 may be closed so as to secure the temperature pack within one or more of top pocket 101, first side pocket 102, and/or second side pocket 103. In embodiments with a closeable design, such closeable elements as a button and button hole, zipper, hook and loop fasteners, laces, or other similar elements may be used to removably retain one or more temperature packs within one or more of top pocket 101, first side pocket 102, and/or second side pocket 103.

In embodiments wherein one or more temperature packs are permanently enclosed within one or more of top pocket 101, first side pocket 102, and/or second side pocket 103, it is contemplated that the entire present invention, including the one or more temperature packs within one or more of top pocket 101, first side pocket 102, and/or second side pocket 103, may be externally conditioned, for example, placed in a hot bath or a freezer, so that the temperature pack may attain the desired temperature.

In some embodiments, flexible connection 104 may be composed of a series of hinged or movably connected pieces. In some embodiments, flexible connection 104 may be composed of a flexible material, such as cloth, plastic, elastomer, or any other element capable of flexibility. In some embodiments, flexible connection 104 may be an adjustable element, such that it can be adjusted for size and then remains at that position. In some embodiments, flexible connection 104 may be omitted entirely, so that the present invention may be more rigid, if such properties are desirable.

It is contemplated that interior surface 105 and webbing 106 may both me comprised of an element that is pleasing to the touch, such as cotton, another soft fibrous element, or a type of cushion. In some embodiments, however, it is conceivable that interior surface 105 and webbing 106 may be comprised of a coarser material, or a material chosen for therapeutic or medicinal purposes.

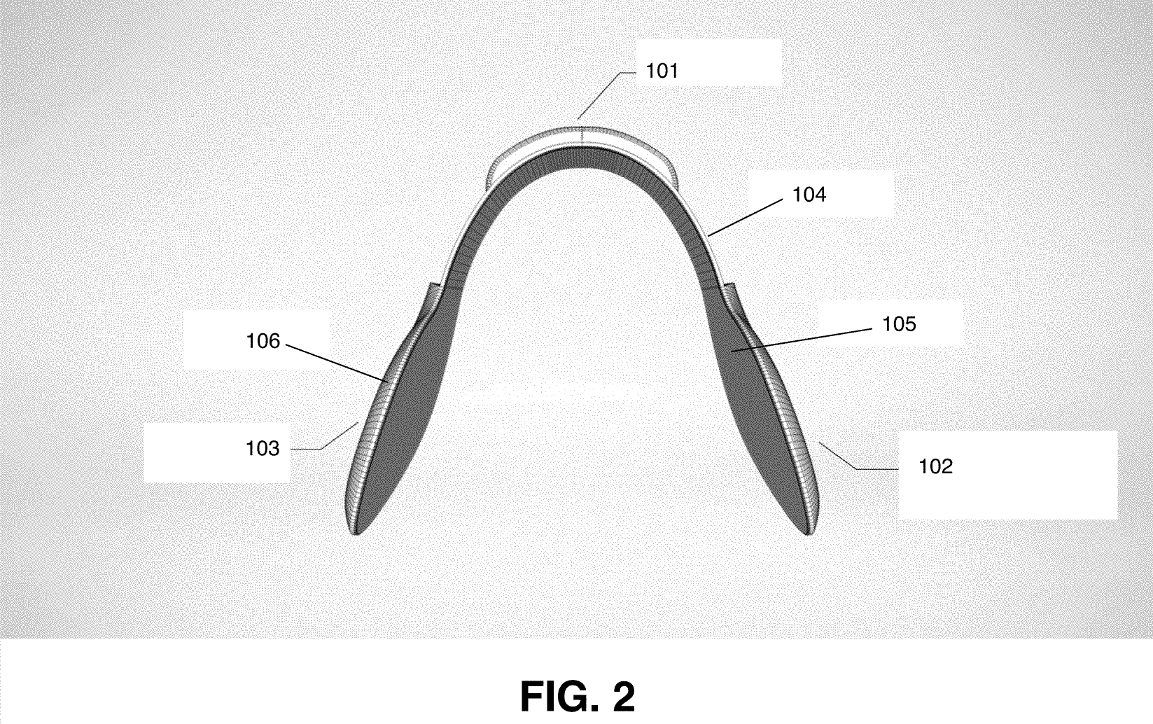

With respect to FIG. 2, a front view of a facial temperature pack holder in accordance with one embodiment of the invention is shown. In the embodiment depicted, a viewer may perceive top pocket 101, first side pocket 102, second side pocket 103, flexible connection 104, interior surface 105, and webbing 106.

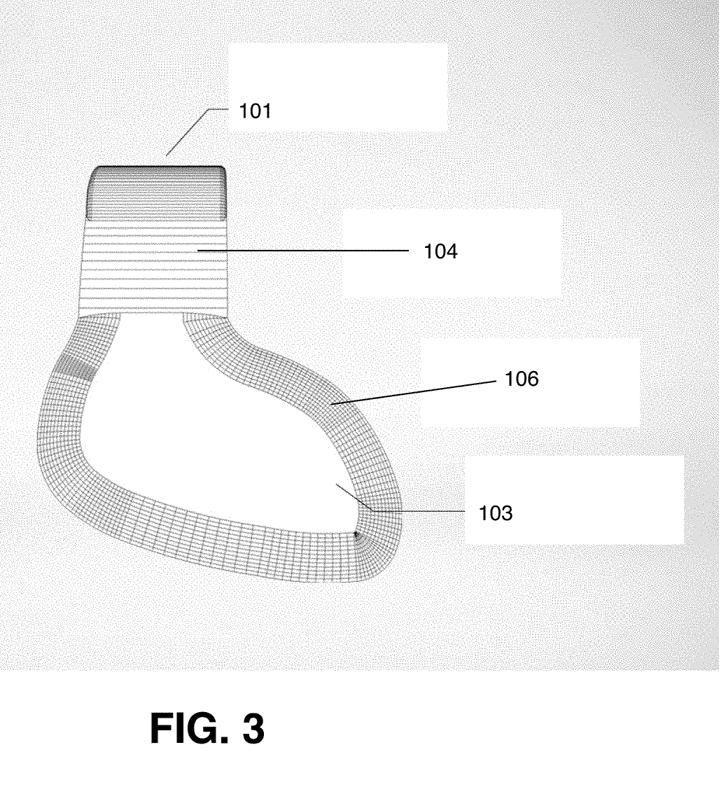

Turning attention to FIG. 3, a side view of a facial temperature pack holder in accordance with one embodiment of the invention is shown. In the embodiment depicted, a viewer may perceive top pocket 101, second side pocket 103, flexible connection 104, and webbing 106.

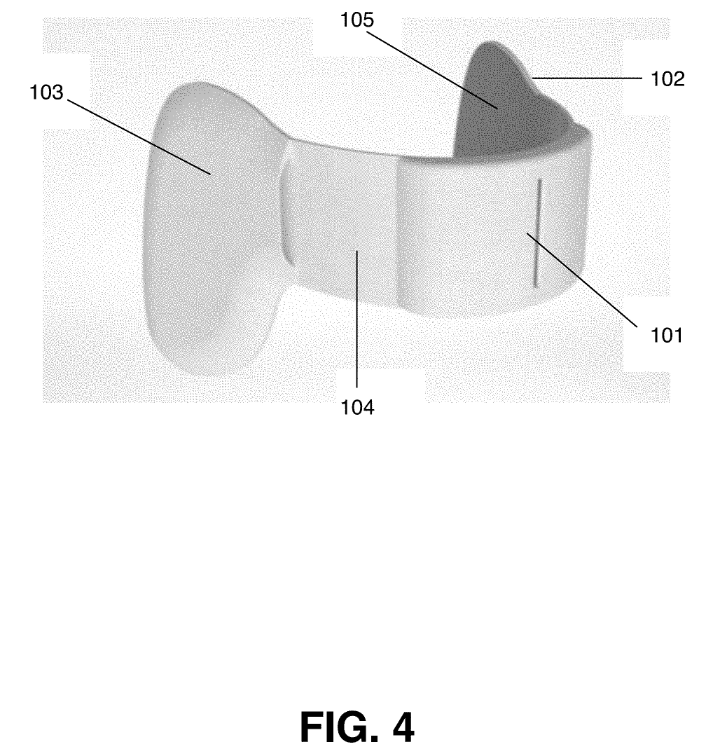

With respect to FIG. 4, a top perspective view of a facial temperature pack holder in accordance with one embodiment of the invention is shown. In the embodiment depicted, a viewer may perceive top pocket 101, first side pocket 102, second side pocket 103, flexible connection 104, and interior surface 105.

In the embodiment disclosed in FIG. 4, a viewer may perceive an embodiment lacking webbing 106. In some embodiments, this may reflect a permanent temperature design wherein the temperature pack is permanently connected to the inside of one or more of top pocket 101, first side pocket 102, and/or second side pocket 103.

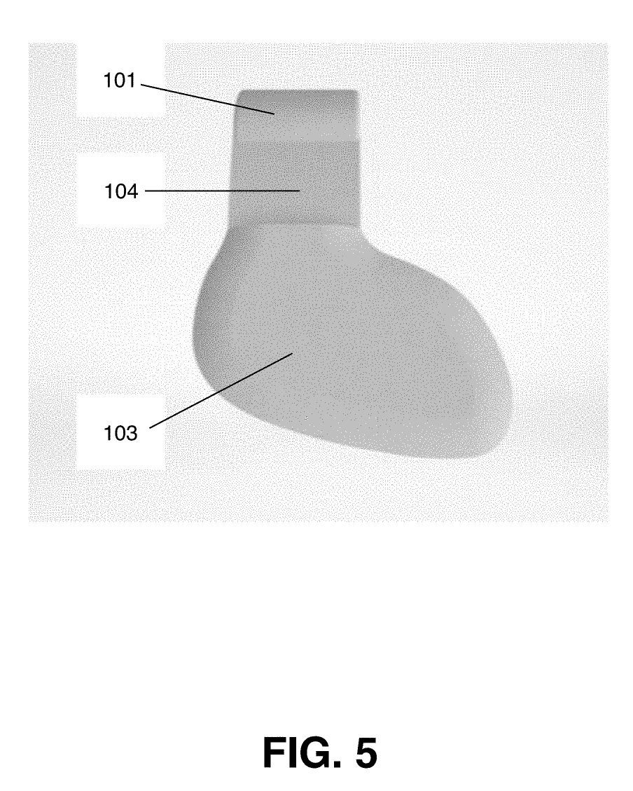

Turning attention to FIG. 5, a top perspective view of a facial temperature pack holder in accordance with one embodiment of the invention is shown. In the embodiment depicted, a viewer may perceive top pocket 101, second side pocket 103, and flexible connection 104.

In the embodiment disclosed in FIG. 5, a viewer may perceive an embodiment lacking flexible connection 104 and webbing 106. In embodiments lacking flexible connection 104 and webbing 106, such as generally and non-limitingly disclosed in FIG. 5, the present invention may be configured such that one or more temperature packs are permanently connected to or inserted within the inside of one or more of top pocket 101, first side pocket 102, and/or second side pocket 103, and the present invention may be rigid, owing to the omission of flexible connection 104.

### CONCLUSIONS, RAMIFICATIONS, AND SCOPE

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

While the temperature pack and holder generally described herein has been disclosed in connection with a number of embodiments shown and described in detail, various modifications should be readily apparent to those of skill in the art.

What is claimed is:

1. A facial temperature pack holder, comprising:
a housing having at least a top portion, a first side portion, and a second side portion;
a top pocket, wherein the top pocket is fixedly connected to the top portion;
a first side pocket, wherein the first side pocket is fixedly connected to the first side portion;
a second side pocket, wherein the second side pocket is fixedly connected to the second side portion; and
at least one temperature pack positioned within the first side pocket.

2. The facial temperature pack holder of claim 1, wherein the at least one temperature pack is removably positioned within the first side pocket.

3. The facial temperature pack holder of claim 1, further comprising at least one temperature pack removably positioned within the second side pocket.

4. The facial temperature pack holder of claim 1, further comprising at least one temperature pack removably positioned within the top pocket.

5. The facial temperature pack holder of claim 1, further comprising at least one flexible connection, wherein the flexible connection is fixedly connected to the top portion and the first side portion.

6. The facial temperature pack holder of claim 1, further comprising at least one flexible connection, wherein the flexible connection is fixedly connected to the top portion and the second side portion.

7. The facial temperature pack holder of claim 1, wherein the first side portion is configured to cover at least part of a facial cheek.

8. The facial temperature pack holder of claim 1, wherein the second side portion is configured to cover at least part of a facial cheek.

9. The facial temperature pack holder of claim 1, wherein the housing is shaped like earmuffs, and wherein the shape of the housing is configured for use with facial cheeks.

10. The facial temperature pack holder of claim 1, wherein the at least one temperature pack is permanently affixed within the first side pocket.

11. The facial temperature pack holder of claim 1, further comprising at least one temperature pack permanently affixed within the second side pocket.

12. The facial temperature pack holder of claim 1, further comprising at least one temperature pack permanently affixed within the top pocket.

13. The facial temperature pack holder of claim 1, further comprising an interior lining on the inside of the housing.

14. The facial temperature pack holder of claim 1, further comprising at least one webbing element disposed along at least one outside edge of the housing.

15. The facial temperature pack holder of claim 1, wherein the housing is shaped like headphones, and wherein the shape of the housing is configured for use with ears.
